(19) United States
(12) Patent Application Publication
(10) Pub. No.: US 2012/0067345 A1
(43) Pub. Date: Mar. 22, 2012

(54) Nasal Dilation Method And Device

(76) Inventor: Eyal Shilon, Tel Aviv (IL)

(21) Appl. No.: 12/888,078

(22) Filed: Sep. 22, 2010

(51) Int. Cl. A61M 29/00; A61M 16/00

(52) U.S. Cl. 128/203.12; 606/199; 604/103.02; 128/200.24

(57) ABSTRACT

A nasal dilator adapted to stretch facial skin, including a resilient layer and attachment means. The resilient layer has an equilibrium configuration and a plurality of nonequilibrium configurations. Portions of the resilient layer are disposed upon a plurality of points upon the facial skin. The facial skin may be stretched by attaching the nasal dilator in a nonequilibrium configuration to the facial skin by the attachment means and allowing the nasal dilator to stretch the facial skin as the resilient layer moves towards the equilibrium.

### BACKGROUND

1. Technical Field

Embodiments of the present invention relate generally to nasal dilation and, more particularly, to a device and method for increasing efficiency and comfort of breathing.

2. Description of Related Art

The use of strips or bands to distort the nose surface, and thereby change the shape of nasal cavities, has been found to be an effective aid to increase the cross sectional area of nasal passages and thereby increase the respiratory efficiency of the user's breathing cycle. Those suffering from allergies, sleep apnea, and other upper respiratory ailments may all benefit from use of devices that help increase the inspiratory and expiratory flow rates.

For example, U.S. Pat. No. 5,806,525 discloses an apparatus for opening nasal passages of the human head. The apparatus includes a metallic biasing member having a length sufficient to span the distance from the first nasal wall to the second nasal wall across the bridge portion. The apparatus further includes a base secured to the biasing member, having a length sufficient to span a distance from the first cheek to the second cheek across the nose. Additionally, the apparatus includes an adhesive material disposed on the biasing member and the base for attaching the biasing member and the base to the nose and cheeks. When the device is attached to the nose and cheeks, the biasing member pulls outwards on the nostrils, increasing the nostril opening diameter and thereby increasing the breathing efficiency.

However the device is designed to apply outward forces for increasing the nostril diameter, and does not employ any other regions for purposes of aiding breathing. The base spanning the cheeks is used for support purposes and not for purposes of changing the facial morphology.

Hence, an improved method for changing the overall facial morphology by means of elastic or other biasing members is still a long felt need.

### BRIEF SUMMARY

According to an aspect of the present invention, there is provided a system and method for increasing the respiratory efficiency and inspiratory and expiratory flow rates by means of providing controlled changes in skin tension on the face.

An aspect of the present invention provides a nasal dilator adapted to stretch facial skin, the dilator including a resilient layer and attachment means, said resilient layer having an equilibrium configuration and a plurality of nonequilibrium configurations, portions of said resilient layer being disposed upon a plurality of points upon said facial skin. The facial skin may be stretched by attaching said nasal dilator in a nonequilibrium configuration to said facial skin by said attachment means and allowing said nasal dilator to stretch said facial skin as said resilient layer moves towards said equilibrium.

It is within provision of the invention to provide a method for aiding breathing of a user, comprising:
a. providing a nasal dilator adapted to stretch facial skin comprising: a resilient layer, said resilient layer having an equilibrium configuration and at least one nonequilibrium configuration; and, attachment means adapted to attach said resilient layer to said users face;
b. deforming said nasal dilator to one said non-equilibrium configuration;
c. attaching said nasal dilator to said user's cheeks; and
d. releasing said nasal dilator, thereby allowing said nasal dilator to exert forces towards said equilibrium configuration, and thus stretching said user's cheeks.

Various embodiments of the invention provide different facial deformations, such that a variety of changes in the nasal cavity membrane configuration can be achieved.

For example, the aforementioned adhesive layer need not necessarily be adhesive; as will be clear to one skilled in the art, any attachment means appropriate may be used. For example, a layer providing enough friction against the skin that it does not slip would suffice instead of an adhesive layer. Alternatively, the device may be anchored upon any appropriate protruberance of the head, for example against the nose, neck, eyesocket, or the like.

Another aspect of the present invention provides a method for aiding breathing of a user, the method including: providing a nasal dilator adapted to stretch facial skin comprising: a resilient layer, said resilient layer having an equilibrium configuration and at least one nonequilibrium configuration; and, attachment means adapted to attach said resilient layer to said users face; deforming said nasal dilator to one said non-equilibrium configuration; attaching said nasal dilator to said user's cheeks; and releasing said nasal dilator, thereby allowing said nasal dilator to exert forces towards said equilibrium configuration, and thus stretching said user's cheeks.

These, additional, and/or other aspects and/or advantages of the present invention are: set forth in the detailed description which follows; possibly inferable from the detailed description; and/or learnable by practice of the present invention.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which:

FIGS. 1A-1H illustrate nasal dilation apparatuses consistent with embodiments of the invention, wherein FIG. 1A illustrates a three-layer embodiment of the device;

FIG. 1B depicts a two-layer embodiment of the device from side view;

FIG. 1C depicts a two-layer embodiment of the device from perspective view;

FIG. 1D depicts a curvilinear embodiment of the device from side view;

FIG. 1E depicts a linear embodiment of the device having bent ends from side view;

FIG. 1F depicts an omega-shaped embodiment of the device having bent ends from side view;

FIG. 1G depicts the omega-shaped embodiment of FIG. 1F after deployment;

FIG. 1H depicts an embodiment of the device that makes use of a rod-shaped resilient member; and

FIG. 2 illustrates the deflection of a cantilever.

### DETAILED DESCRIPTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for providing a nasal dilator.

In the following detailed description, numerous specific details are set forth in order to provide a thorough understanding of embodiments of the present invention. However, those skilled in the art will understand that such embodiments may be practiced without these specific details. Reference throughout this specification to “one embodiment” or “an embodiment” means that a particular feature, structure, or characteristic described in connection with the embodiment is included in at least one embodiment of the invention.

The term ‘plurality’ refers hereinafter to any positive integer (e.g, 1, 5, or 10).

The term ‘deforming strip’ refers hereinafter to the device of the current invention.

The term ‘spring-like’ refers hereinafter to the property of resilience, whereby an object when deformed from an equilibrium state tends to return to the equilibrium state or to one of several equilibrium states. For example, a torsion spring has resilient characteristics tending to return it to an equilibrium state upon deformation.

It has long been known that the inspiratory and expiratory flow rates of human breathing are susceptible to the effects of changes in the sinus passage and mucous membrane configurations. For example, the simple expedient of flaring the nostrils outwards can increase flow rates substantially. For this reason runners and racehorses will both be observed to flare their nostrils during competition.

As will be appreciated from the anatomy of the sinus, the nostril constitutes but the very beginning of a complex series of passages leading from the nose to the rest of the upper respiratory tract. Any number of these passages may become constricted or blocked by mucous membrane inflammation, the inherent anatomy, various surgeries or trauma, and the like. However many of these passages may also be somewhat deformed by deformation of the overlying skin tissue, including but not limited to the atrium, middle and inferior meatus, and vestibule. The musculature of the face, as those skilled in the art will appreciate, suggests that significant effects for sinus blockage may be produced by application of force upon the correct muscle groups.

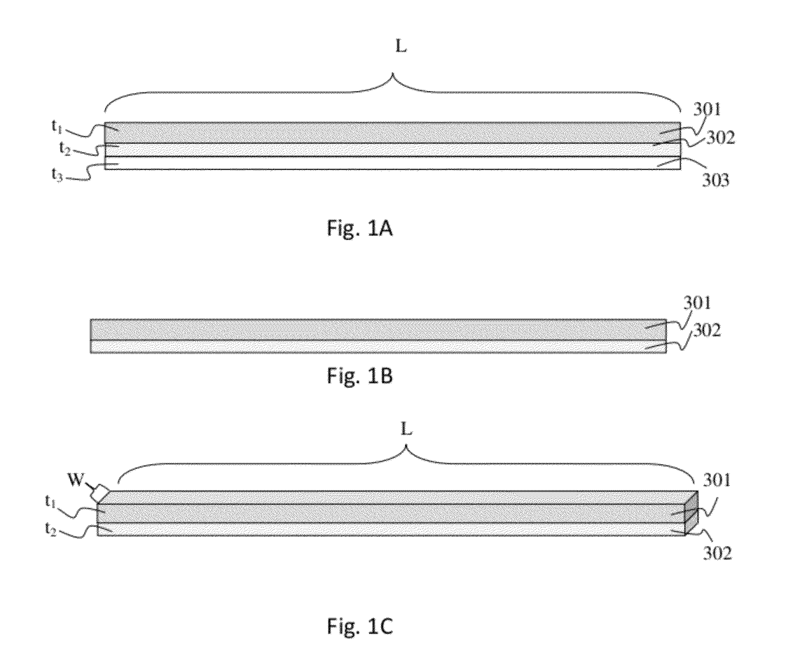

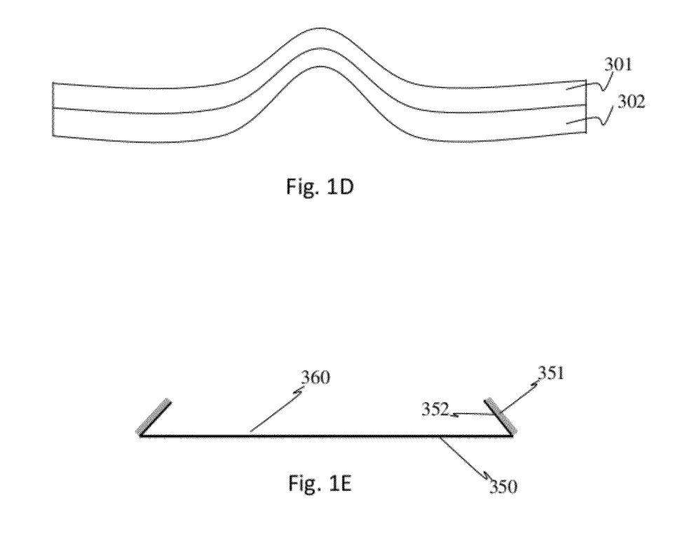

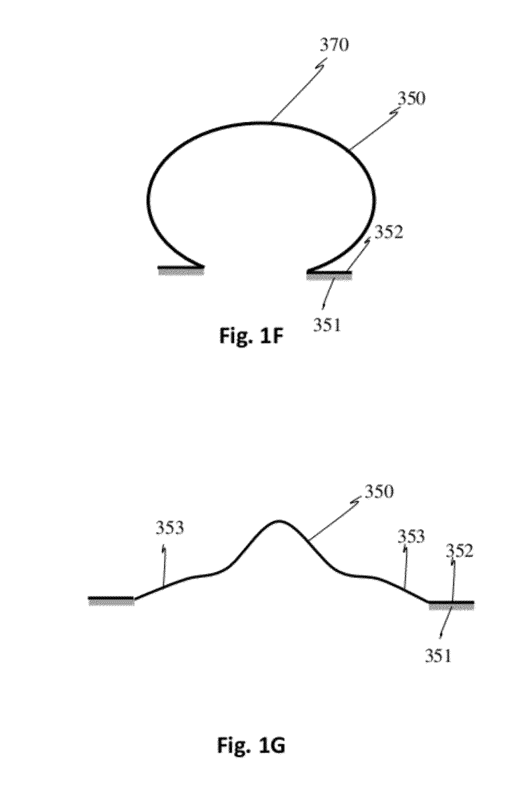

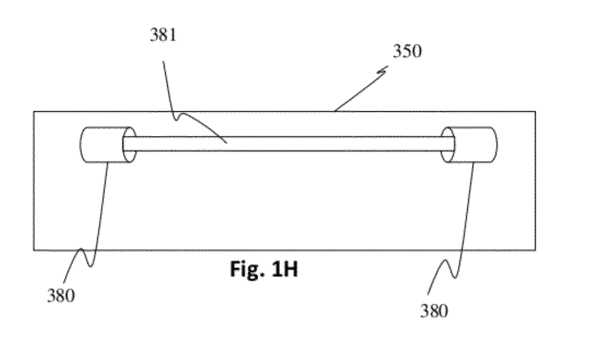

In a preferred embodiment of the invention, a deforming strip is provided comprising several layers. In one embodiment of the invention a three-layer structure is used. As shown in an exemplary embodiment in FIG. 1a, a bottom adhesive-protection layer 303 is provided, which covers and protects an adhesive layer 302 that is in turn attached to a flexible layer 301 having resilient spring-like characteristics.

When the device is to be used, the adhesive-protection layer 303 is removed as shown in FIG. 1b, leaving the adhesive layer 302 and the spring-like layer 301. As will be appreciated by one skilled in the art the adhesive layer may be rendered unnecessary by use of other attachment means.

As will be appreciated by one skilled in the art, the use of the adhesive-protection layer is not necessary for operation of the device; for instance an adhesive layer 302 may be employed that only becomes adhesive under certain conditions (e.g. of temperature, pressure, etc.), or that only adheres to human skin.

The operation of the device relies on the spring-like characteristics of the spring-like layer 301. When the device is bent into a configuration such as that shown in FIG. 1d, the spring-like layer will tend to return to its equilibrium state, which in this example is the flat state shown in FIG. 1b. If the adhesive layer is attached to the skin in a non-equilibrium configuration such as that shown in FIG. 1d, then the return of the device to a configuration closer to the flat equilibrium position of FIG. 1b will tend to pull the skin attached to the adhesive layer along with it, in effect stretching the skin to some new position dependent on the initial state of the device, the spring characteristics of the spring-like layer, and the adhesive properties of the adhesive layer.

As shown in FIG. 1a, the thicknesses of three layers of the device are denoted t₁, t₂, and t₃. The lengths of the layers in some embodiments are equal, being denoted here as L (FIG. 1c). The layers of the device in some embodiments have rectangular cross sections, and thus the third dimension perpendicular to the page in FIG. 1c is referred to hereinafter as the width W, which in some embodiments is common to all layers of the device.

In another embodiment of the device illustrated in FIGS. 1e, f, g, two adhesive strips 351 are employed at the ends 352 of a resilient or spring-like substrate 350. This substrate is in its equilibrium state 360 largely linear as shown in FIG. 1e, but for the ends 352 which are bent as shown in the figure. The entire spring-like substrate 350 is bent by the user into an omega-shaped form 370 as shown in FIG. 1f, from which the resilient member will have a tendency to return to its equilibrium state of FIG. 1e. The user bends the device into the omega shape and uses the adhesive strips 351 to adhere the device to the cheeks, with the substrate 350 spanning the nose or other area. When released, the tendency of the spring-like resilient substrate 350 to attempt to return to its equilibrium state will exert a pressure upon the skin attached to the adhesive strips tending to pull these areas in opposing directions. One possible relaxed state is shown in FIG. 1g, for the case where the device has been put into use over a user's nose. As can be seen from the figure, the resilient strip has pushed the areas 352 (that are attached to the cheeks by adhesive means) as well as intervening areas 353 outwards, thereby effectively pushing the cheeks towards the ears, dilating the nostrils and allowing easier breathing. Another possible final state is that shown in FIG. 1f, where the omega shape is large enough to clear the nose entirely, not touching it but rather touching the face only at the areas 352 having adhesive strips 351.

A further embodiment of the invention is shown in FIG. 1h. A set of holders 380 is provided. A flexible rod 381 is inserted into the cup-like holders 380. These holders may have any suitable form, variations of which will be obvious to one skilled in the art. The rod provides resilience to the device, which is further provided on the underside with adhesive strips (not seen) or other attachment means in order to attach the device to the cheeks. The advantage of this embodiment is that a set of rods 381 may be provided each with a different spring constant such that the user can choose the rod most effective for him or her. In effect, this embodiment allows for a variable spring constant to be chosen by the user.

The rod may be further used to hold and deliver material, such as medication, scents, nasal-passage opening materials, and the like. The rod can for instance be provided with stoppers or caps at the ends, which are removed just before use. The medication or other material within the rod, which may be liquid, gel, or other phase of matter, may then seep out the ends of the rod and onto small perforations within the substrate 350. Alternatively, the rod may have holes in its sides, and/or the cup-like holders 380 may be provided with mesh or perforations to allow for the medication or other material to diffuse out of the rod, onto the substrate, through the substrate, and onto the skin of the users' cheeks. Alternatively, the substrate material and/or the attachment means can be used for delivery of such substances as are described above, for example the substrate material may be impregnated with medication.

Furthermore this rod may be itself composed of medication or other material that may have effects for the user, such as Eucalyptus scent, aromatic salts, vasoconstrictive fluids, or the like. Alternatively the rod may be hollow and filled by the material to be dispersed. A similar embodiment has the rod surrounded by the material to be dispersed. Further embodiments will be clear to one skilled in the art. The aforementioned rod may be selected from a set, each for example with a different material and/or spring constant of resilience, as discussed below.

It will be appreciated by one skilled in the art that this embodiment effectively uses the resilient member under compression, and not under tension as in the prior art.

It is within provision of the invention to provide strips that have a cantilever spring constant as defined by the equation

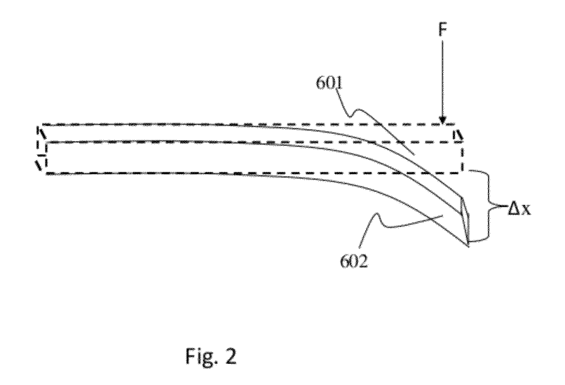

F=kΔx,

where F is the force perpendicular to the cantilever, k is the spring constant, and Δx is the deflection from the equilibrium position, as shown in FIG. 2, where the force is perpendicular to the cantilever deflection. The undeflected cantilever 601 is shown in dotted outline whereas the deflected cantilever 602 is shown in solid outline. The force constant k may in some embodiments preferably be between 1 N/m and 1000 N/m.

For embodiments of the invention using an adhesive layer as opposed to other attachment means, the adhesive properties of the adhesive layer are important to proper operation of the device, since a weak adhesion will allow the device to detach from the skin, while too strong an adhesion will cause discomfort and/or injury upon removal. Thus definition of the pull-off stress (the pressure required to pull the adhesive layer off a substrate) must be addressed. The adhesive strength is hereinafter defined to be the maximum pressure (force per unit area) that may be applied to pulling the adhesive layer adhering to a substrate, before the adhesive layer pulls of the substrate, according to the equation

$$P = \frac{F}{A} ,$$

where P is the pressure, F is the force applied at the moment of detachment, and A is the area of the adhesion zone.

In some embodiments of the invention, the adhesive strength of the adhesive layer is between 100 Pa and 10,000 Pa, where the Pascal (Pa) is 1 N/m².

As will be appreciated by one skilled in the art, the deforming strip of the current invention will find use in a variety of anatomical situations, and is thus able to affect inter alia the nasal turbinates, the nasal pharynx, the oral pharynx, and the nasal mucosa.

It is within provision of the invention that a long strip bridging the nose be used. In one such case the strip bridges the nose and attaches to the cheeks, stretching the cheek muscle and deforming the underlying nasal structures as described above.

It is further within provision of the invention that the substrate layer be divided into several sections, for example being divided into two sections each of which attaches to the face at different locations, these locations being stretched relatively by means of (for example) a rod connecting the disparate sections of the substrate.

It is further within provision of the invention to use a plurality of such rods, for example two or more rods being connected in a network with the ends of each rod being attached to substrate pads. These pads are ideally designed to comfortably press upon the skin and stretch the skin due to the forces upon the rod or rods attached to it.

It is within provision of the invention to stretch only one cheek or any other single point upon the face. This may be accomplished for instance by use of two or more attachment pads instead of a complete strip. One pad is attached for instance to one end of a resilient rod and placed upon a cheek or other facial area to be stretched. The other end of the resilient rod is attached to another pad, which is anchored for instance against the nose, eye socket, jaw or other appropriate location. By compressing the rod before attachment, a sensible pressure will be exerted upon the rod between tending to force the pads in a direction determined by their geometrical configuration and the disposition of the rod.

It should be appreciated that unlike the nasal dilators seen in the prior art, the nasal dilator of the current invention may operate, in certain embodiments, in a mode of compression; the prior art nasal dilators employ resilient members that are under tension only.

It should further be appreciated that the skin of the user's face may be pulled in many different ways depending upon the specific embodiment deployed. For instance, use of a dilator with a linear equilibrium configuration and an omega-shaped pre-deployment configuration will tend to push those areas attached to the ends of the omega shape, outwards. Alternatively use of an omega-shaped equilibrium configuration with a linear pre-deployment configuration will tend to pull those areas attached to the ends of the omega shape, inwards. By so doing the skin of the face may be either pulled towards the nose or towards the ears, if for example the cheeks are used as attachment points.

All examples discussed herein are non-limiting examples.

The disclosed embodiments may be variously combined.

Although selected embodiments of the present invention have been shown and described, it is to be understood the present invention is not limited to the described embodiments. Instead, it is to be appreciated that changes may be made to these embodiments without departing from the principles and spirit of the invention, the scope of which is defined by the claims and the equivalents thereof.

What is claimed is:

1. A nasal dilator adapted to stretch facial skin, comprising a resilient layer and attachment means, said resilient layer having an equilibrium configuration and a plurality of nonequilibrium configurations, portions of said resilient layer being disposed upon a plurality of points upon said facial skin, wherein facial skin may be stretched by attaching said nasal dilator in a nonequilibrium configuration to said facial skin by said attachment means and allowing said nasal dilator to stretch said facial skin as said resilient layer moves towards said equilibrium.

2. The nasal dilator of claim 1, wherein said plurality of points are selected from the group consisting of: the cheek, the eyesocket, the nose, the chin, the forehead, the mouth, the septum.

3. The nasal dilator of claim 1, wherein said attachment means comprise an adhesive layer.

4. The nasal dilator of claim 1, further comprising an adhesive protection layer.

5. The nasal dilator of claim 1 wherein said resilient layer comprises a plurality of rod holders and a plurality of flexible rods attached to said rod holders providing resilience.

6. The nasal dilator of claim 1, wherein said resilient layer is adapted to contain therapeutic material, and wherein said resilient layer is adapted to release said material.

7. The nasal dilator of claim 6 wherein said therapeutic material is selected from the group consisting of: medication, scents, nasal-passage opening materials, eucalyptus oil, tea tree oil, lavender oil, aromatherapy oils, aromatic compounds, phytoncides, herbal distillates, aqueous plant extracts, saline solution, antibiotics, sodium bicarbonate solution, xylitol, alcohol, and combinations thereof.

8. The nasal dilator of claim 1, wherein said resilient layers comprise therapeutic material, and wherein said resilient layer is adapted to diffuse said material.

9. The nasal dilator of claim 1, wherein said equilibrium configuration is of a geometric form selected from the group consisting of: V-shaped, U-shaped, W-shaped, omega-shaped.

10. The nasal dilator of claim 1, wherein said resilient layer has a spring constant of between 1 N/m and 1000 N/m.

11. The nasal dilator of claim 1, wherein said adhesive layer has an adhesive strength of between 10,000 Pa and 100 Pa.

12. A method for aiding breathing of a user, comprising:
a) providing a nasal dilator adapted to stretch facial skin comprising: a resilient layer, said resilient layer having an equilibrium configuration and at least one nonequilibrium configuration; and, attachment means adapted to attach said resilient layer to said users face;
b) deforming said nasal dilator to one said non-equilibrium configuration;
c) attaching said nasal dilator to said user's cheeks; and
d) releasing said nasal dilator, thereby allowing said nasal dilator to exert forces towards said equilibrium configuration, and thus stretching said user's cheeks.

13. The method of claim 12, wherein said attachment means comprises an adhesive layer.

14. The method of claim 13, further comprising an adhesive protection layer.

15. The method of claim 12, wherein said resilient layer further comprises a plurality of rod holders and a plurality of flexible rods attached to said rod holders providing resilience.

16. The method of claim 12, wherein said resilient layer is adapted to contain therapeutic material, and wherein said resilient layer is adapted to release said material.

17. The method of claim 16 wherein said therapeutic material is selected from the group consisting of: medication, scents, nasal-passage opening materials, eucalyptus oil, tea tree oil, lavender oil, aromatherapy oils, aromatic compounds, phytoncides, herbal distillates, aqueous plant extracts, saline solution, antibiotics, sodium bicarbonate solution, xylitol, alcohol, and combinations thereof.

18. The method of claim 12, wherein said equilibrium configuration is of a geometric form selected from the group consisting of: V-shaped, U-shaped, W-shaped, omega-shaped.

19. The method of claim 12, wherein said resilient layer has a spring constant of between 1 N/m and 1000 N/m.

20. The method of claim 11, wherein said adhesive layer has an adhesive strength of between 10,000 Pa and 100 Pa.
